(19) United States
(12) Patent Application Publication
(10) Pub. No.: US 2021/0005309 A1
(43) Pub. Date: Jan. 7, 2021

(54) METHOD AND SYSTEM FOR MANAGING SUPPLIER FOR HEALTHCARE ORGANIZATIONS

(71) Applicant: Premier Healthcare Alliance, LP, Charlotte, NC (US)

(72) Inventors: Mickey Meehan, Walnut Creek, CA (US); Bill Sambrone, Walnut Creek, CA (US); Nicole Barker, Walnut Creek, CA (US); Anna Ormiston, Walnut Creek, CA (US)

(73) Assignee: Premier Healthcare Alliance, LP, Charlotte, NC (US)

(21) Appl. No.: 16/918,075

(22) Filed: Jul. 1, 2020

Related U.S. Application Data
(60) Provisional application No. 62/869,398, filed on Jul. 1, 2019.

(51) Int. Cl. G16H 40/20

(52) U.S. Cl. CPC G16H 40/20

(57) ABSTRACT

A system for managing suppliers for managing source entities for recipient entities includes a source entity evaluation system, and a category file database storing a plurality of category files having a plurality of amount values. The system further comprises a source entity management database storing a plurality of complexities for the source entities and configured to received updated complexities data from a source entity system. Moreover, a benchmarking module stores a plurality of source entity quality standards to be bases of the amount values and configured to generate maturity amount values in accordance with the amount values and the source entity quality standards. The system further comprises a cost analysis and an electronic record management module.

### CROSS-REFERENCE TO PRIORITY APPLICATION

This application claims priority to and the benefit of U.S. Provisional Patent Application Ser. No. 62/869,398, filed Jul. 1, 2019 entitled “Method and System for managing supplier for healthcare organizations,” which is hereby incorporated by reference in its entirety.

### FIELD OF THE DISCLOSURE

The present invention relates to methods, systems, and computer program products for managing source entities for recipient entities. In some instances, the invention involves evaluating data associated with source entities to determine resource providers for recipient entities.

### BACKGROUND

Recipient systems/entities typically require transmissions of a variety of resources such as components, items, devices, services, etc. The important consideration in the resource provider qualification is quality and reliability. However, conventional systems for resource provider qualification have several drawbacks. Conventional systems are not structured for direct operative communication between recipient entities and the source entities. Therefore, there is a need to for methods, systems, and computer program products for managing source entities, establishing direct operative communication between recipient entities and the source entities, and evaluating data associated with source entities to determine resource providers for recipient entities, without invoking undue complexities for use and without requiring expensive equipment.

All referenced patents, applications, and literature are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The disclosed embodiments may seek to satisfy one or more of the above-mentioned desires. Although the present embodiments may obviate one or more of the above-mentioned desires, it should be understood that some aspects of the embodiments might not necessarily obviate them.

### BRIEF SUMMARY

The following presents a simplified summary of one or more embodiments of the invention in order to provide a basic understanding of such embodiments. This summary is not an extensive overview of all contemplated embodiments, and is intended to neither identify key or critical elements of all embodiments, nor delineate the scope of any or all embodiments. Its sole purpose is to present some concepts of one or more embodiments in a simplified form as a prelude to the more detailed description that is presented later.

Embodiments of the present invention provide a method, system and computer program product for managing source entities. In some instances, the invention involves evaluating quality data associated with source entities in order to determine one or more resource providers out of the source entities, for healthcare organizations. Specifically, embodiments of the present invention provide a method, system and computer program product for managing source entities for recipient entities comprising healthcare organizations. In some embodiments, the system comprises a source entity evaluation system, comprising; a category file database storing a plurality of category files having a plurality of amount values; a source entity management database storing a plurality of complexities for the source entities and configured to received updated complexities data from a source entity system; a benchmarking module storing a plurality of source entity quality standards to be bases of the amount values and configured to generate maturity amount values in accordance with the amount values and the source entity quality standards; a cost analysis module configured to calculate amounts of expenses for each of the source entities to generate the amount values; and an electronic record management module storing a plurality of maturity electronic records having the plurality of category files having the maturity amount values.

In some embodiments, or in combination with any of the previous embodiments, the source entity system provides category files to the source entity evaluation system through a communication network.

In some embodiments, or in combination with any of the previous embodiments, an entity terminal system provides user's information and user's electronic records to the source entity evaluation system through the communication network.

In some embodiments, or in combination with any of the previous embodiments, the source entity evaluation system further comprises a negotiate module communicating with the source entity system and the entity terminal system through the communication network, wherein the negotiate module can receive source entity responses in response to category files provided by the source entity system.

In some embodiments, or in combination with any of the previous embodiments, the source entity evaluation system further comprises a predictive plan database to store a plurality of customer's electronic records provided by the entity terminal systems.

In some embodiments, or in combination with any of the previous embodiments, the complexities are selected from the group consisting of locations, services levels, service types, transaction costs, service terms, special discounts, and potential risks.

In some embodiments, or in combination with any of the previous embodiments, the predictive plan supplies the customer's electronic records to the cost analysis module, the amount of values being calculated in accordance with the source entity quality standards in the customer's electronic records.

In some embodiments, or in combination with any of the previous embodiments, the source entity evaluation system further comprises an interface enabling remote access through the communication network.

In some embodiments, or in combination with any of the previous embodiments, the source entity evaluation system further comprises an interactive evaluation module to identify a compliance source entity to the maturity electronic records in accordance with the maturity amount values.

Some embodiments of the invention are directed to a method performed by a computer for managing source entities for recipient entities comprising healthcare organizations. The method comprises the steps of: inputting user information to a source entity evaluation system via an interface; loading a customer's electronic record having a plurality of category files from a predictive plan database; calculating amount values from the customer's electronic record in accordance with source entity quality standards provided by a benchmarking module to generate maturity amount values; and presenting a maturity electronic record by an electronic record management module.

In some embodiments, or in combination with any of the previous embodiments, the method further comprises updating customer's electronic records by a source entity system via a communication network, wherein the electronic records comprise electronic records.

In some embodiments, or in combination with any of the previous embodiments, the method further comprises updating the category files in accordance with a source entity management database.

In some embodiments, or in combination with any of the previous embodiments, the method further comprises negotiating the amount values with the source entities via a negotiate module.

In some embodiments, or in combination with any of the previous embodiments, the method further comprises computing a percentage of the amount values in the category file and identify a critical category file with a larger amount of percentage.

In some embodiments, or in combination with any of the previous embodiments, the method further comprises uploading customer's electronic records provided by an entity terminal system to the predictive plan.

In some embodiments, or in combination with any of the previous embodiments, the method further comprises initiating an interactive evaluation section to select a compliance source entity for the category file.

According to some aspects of the invention, in a general implementation, a computer system for managing suppliers for healthcare organizations includes a supplier evaluation system, having: a category file database storing a plurality of category files having a plurality of amount values; a supplier management database storing a plurality of complexities for the suppliers and configured to received updated complexities from a supplier system; a benchmarking module storing a plurality of supplier quality standards to be bases of the amount values and configured to generate maturity amount values in accordance with the amount values and the supplier quality standards; a cost analysis module to calculate amounts of expenses for each of the suppliers to generate the amount values; and a contract management storing a plurality of maturity contracts having the plurality of category files having the maturity amount values.

In another aspect combinable with the general implementation, the system further comprises a supplier system providing category files to the supplier evaluation system through a communication network.

In another aspect combinable with the general implementation, the system further comprises a business terminal system providing user's information and user's contracts to the supplier evaluation system through the communication network.

In another aspect combinable with the general implementation, the system further comprises a negotiate module communicating with the supplier system and the business terminal system through the communication network, wherein the negotiate module can receive supplier responses in response to category files provided by the supplier system.

In another aspect combinable with the general implementation, the supplier evaluation system further comprises a predictive plan database to store a plurality of customer's contracts provided by the business terminal systems.

In another aspect combinable with the general implementation, at least one of the complexities are selected from the group consisting of locations, services levels, service types, transaction costs, service terms, special discounts, and potential risks.

In another aspect combinable with the general implementation, at least one of the predictive plan supplies the customer's contracts to the cost analysis module, the amount values being calculated in accordance with the supplier quality standards in the customer's contracts.

In another aspect combinable with the general implementation, the supplier evaluation system further comprises an interface enabling remote access through the communication network.

In another aspect combinable with the general implementation, wherein the supplier evaluation system further comprises an interactive evaluation module to identify a compliance supplier to the maturity customer's contracts in accordance with the maturity amount values.

Another aspect of the embodiment is directed to a method for managing suppliers for a healthcare organization, wherein the method comprises the steps of: inputting user information to a supplier evaluation system via an interface; loading a customer's contract having a plurality of category files from a predictive plan database; calculating amount values from the customer's contract in accordance with supplier quality standards provided by a benchmarking module to generate maturity amount values; and presenting a maturity contract by a contract management.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above and below as acting in certain combinations and even initially claimed as such, one or more features from a claimed combination can in some cases be excised from the combination, and the claimed combination may be directed to a sub-combination or variation of a sub-combination.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

Having thus described embodiments of the invention in general terms, reference will now be made to the accompanying drawings, wherein:

FIG. 1 illustrates a processing system environment, in accordance with one embodiment of the invention.

FIG. 2 illustrates a schematic representation of system components for managing supplier for a healthcare organization, in accordance with one embodiment of the invention.

FIG. 3 illustrates a high level process flow 300 for a method and process for managing source entities for recipient entities/systems, in accordance with one embodiment of the invention.

FIG. 4 illustrates a graphical interface representation of a category file of the system of FIG. 2, showing the specification items on the category files.

FIG. 5 illustrates a graphical interface representation of the complexities for the category file of the system of FIG. 2.

FIG. 6 illustrates a graphical representation of electronic record management module of the system of FIG. 2, showing the maturity amount values and the tillable sections.

It should be noted that the drawing figures may be in simplified form and might not be to precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to elements throughout. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein.

The different aspects of the various embodiments can now be better understood by turning to the following detailed description of the embodiments, which are presented as illustrated examples of the embodiments defined in the claims. It is expressly understood that the embodiments as defined by the claims may be broader than the illustrated embodiments described below.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

In some embodiments, an “source entity” or “supplier entity” as used herein may be any institution associated with providing resources. The resources may comprise components, items, devices, services, and/or the like. Each resource may comprise one or more resource attributes such as a resource value. A resource value of a resource may refer to a cost, price and/o the like of the resource. In some embodiments, the “source entity” or “supplier entity” is a supplier associated with providing medical resources such as medical devices, medical equipment, and/or the like. That said, the source entity or supplier entity may be any institution, group, association, establishment, company, union, manufacturer, supplier, seller, and/or the like. In some instances, the present invention may identify a plurality of source entities and evaluate data associated with source entities to determine/assign/qualify at least one source entity as a resource provider (also referred to as a “vendor”) for a recipient entity.

In some embodiments, an “recipient entity” or “buyer entity” as used herein may be any institution associated with receiving/obtaining resources from the source/supplier entity. The source/supplier entity may transfer the resource to the recipient/buyer entity. This resource transfer may also be referred to as a purchase, such that a resource value may be transmitted from the recipient/buyer entity to the source/supplier entity, in exchange for the resource transfer from the source/supplier entity to the recipient/buyer entity. That said, the recipient/buyer entity may be any institution, group, association, establishment, company, union, manufacturer, buyer, intermediate seller, and/or the like. In some instances, the recipient entity is a healthcare organization.

In some embodiments, a “user” as used herein may be any individual or institution associated with receiving resources from the source/supplier entity, e.g., via a resource transfer therebetween. In some embodiments, the “user” is also referred to as a customer of the source/supplier entity (e.g., a supplier). In this regard, the user may be associated with the recipient/buyer entity.

Recipient entities typically require transmissions of a variety of resources such as components, items, devices, services, etc. Generally, recipient entities such as private or public organizations hire purchasing agents to manage resource providers (i.e., vendors) for resources such as high technology goods and services. Here, the purchasing agents would typically evaluate at least one of the source entities (i.e., suppliers) that would qualify as a resource provider (i.e., vendor) who may provide the required/satisfactory resources, such as products and/or level or services, based upon the predetermined project objectives. The important consideration in the resource provider (i.e., vendor) qualification is quality and reliability. The purchasing agents typically provide quotations for the general or specific resources, e.g., services, to the recipient entities (e.g., customers). However, conventional systems for resource provider (i.e., vendor) qualification have several drawbacks. First, conventional systems are not structured for direct operative communication between recipient entities (e.g., customers) and the source entities (i.e., suppliers). Due to this, the recipient entities (e.g., customers) negatively accept the quotations from the purchasing agents without any negotiation with the source entities (i.e., suppliers). Second, the purchasing agents usually incorporate with certain/specific source entities (i.e., suppliers) who can provide them the better offers (e.g., commission), so the database of the source entities (i.e., suppliers) utilized by the purchasing agents is limited.

Therefore, there is a need to for methods, systems, and computer program products for managing source entities, establishing direct operative communication between recipient entities (e.g., customers) and the source entities (i.e., suppliers), and evaluating data associated with source entities to determine resource providers for recipient entities, without requiring undue complexities for use and without requiring expensive equipment. The present invention provides methods, systems, and computer program products for managing source entities, establishing direct operative communication between recipient entities (e.g., customers) and the source entities (i.e., suppliers), and evaluating data associated with source entities to determine resource providers for recipient entities, which is easy to use and inexpensive.

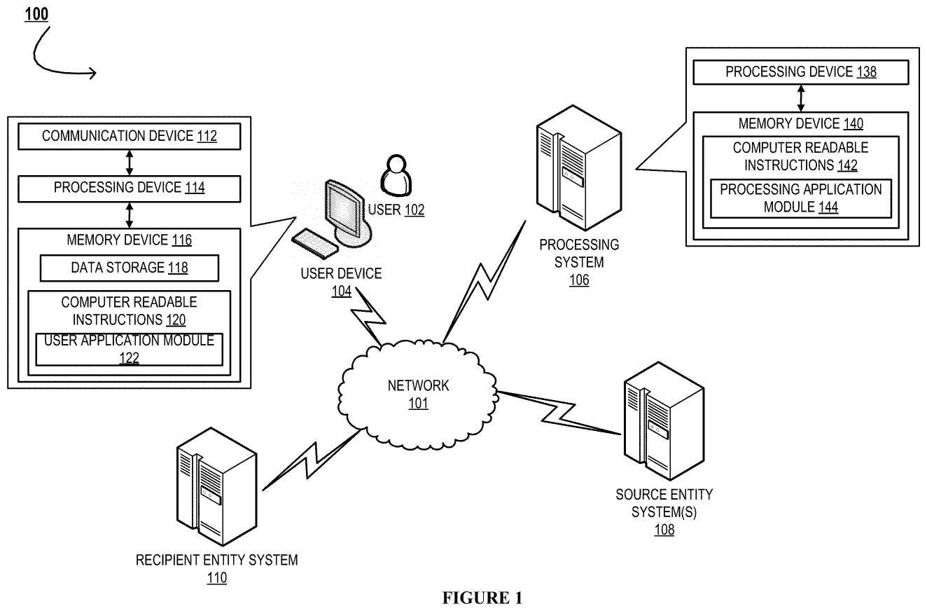

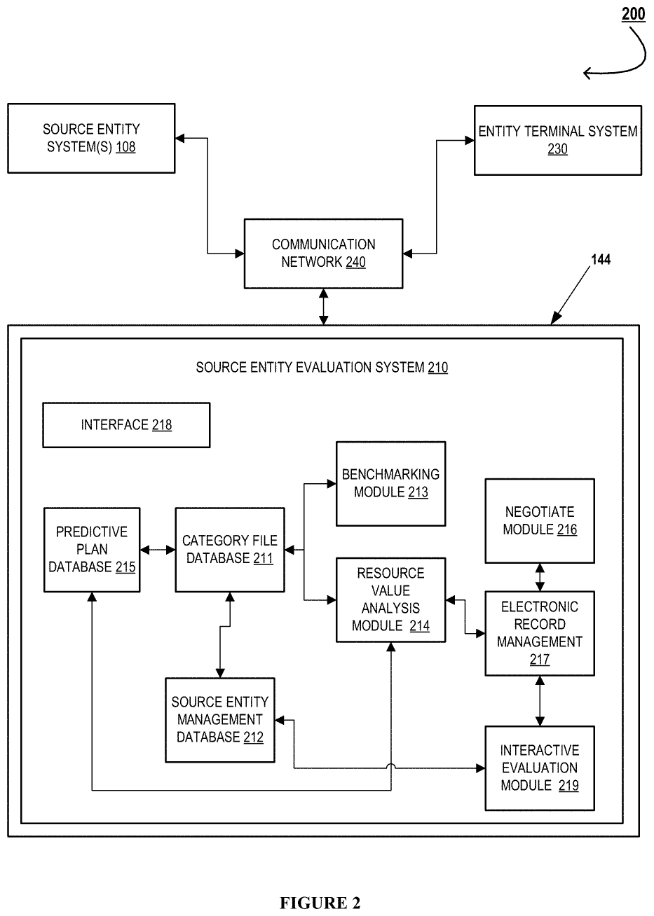

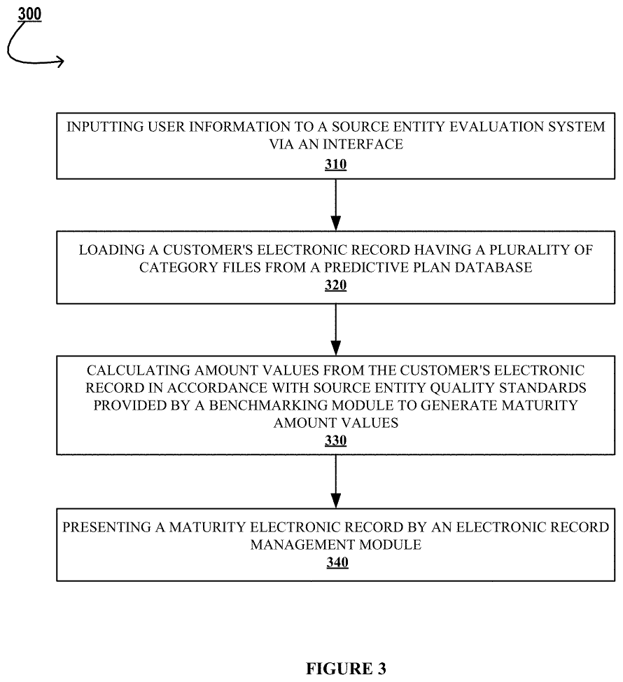

FIG. 1 illustrates a processing system environment 100, in accordance with one embodiment of the invention. As illustrated in FIG. 1, a processing system 106 is operatively coupled, via a network 101 to a user system/device 104 associated with a user 102, a plurality of source entity systems 108 (also referred to as a supplier entity system(s) 108) associated with the source/supplier entities, and/or a recipient entity system 110 (also referred to as a buyer system 110) associated with the recipient/buyer entity such as a healthcare organization. The processing system 106 may also be referred to as a “computing system”, “computing device,” “server” or “system”. The processing system 106 typically comprises at least one processing device 138 that is structured to perform one or more of the steps/functions associated with managing source entities for recipient entities, and evaluating data associated with source entities to determine resource providers for recipient entities (e.g., as described below with respect to FIGS. 2-3). Typically, the at least one processing device 138 is structured to perform one or more of the steps/functions described herein based on executing computer readable instructions/code 142 of a processing application module 144 stored on a memory device 140. The processing system 106 may be in operative communication with and may transmit signals to and receive signals from, the user device 140, the recipient entity system 110 and/or the source entity system 108 associated with the supplier entity. The processing system 106, and the processing application module 144 in particular, is structured for processing resource transfers and constructing resource values, e.g., based on executing computer-readable instructions 142. Specifically, the processing system 106, and the processing application module 144 in particular is structured for processing resource transfers by analyzing purchased services, and more particularly constructing resource values by predicting the prices of the transactions based on the input of the user. FIG. 1 illustrates only one example of an embodiment of the system environment 100, and it will be appreciated that in other embodiments one or more of the systems, devices, or servers may be combined into a single system, device, or server, or be made up of multiple systems, devices, or servers.

The network 101 may be a global area network (GAN), such as the Internet, a wide area network (WAN), a local area network (LAN), near field communication network, audio/radio communication network, ultra-high frequency wireless communication network, or any other type of network or combination of networks. The network 101 may provide for wireline, wireless, or a combination wireline and wireless communication between devices on the network 101.

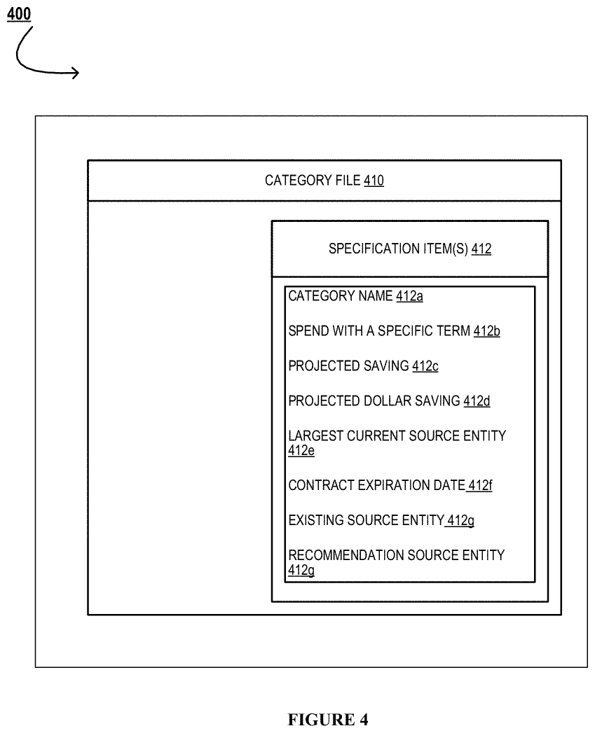

The user device 104 may comprise a communication device 112, a processing device 114, and a memory device 116. The memory device 116 may comprise a data storage 118, along with computer readable instructions 120 for a user application 122. The processing system 106 may transmit control signals/instructions to the user device 104, via the network 101, to cause the user application 122 to display a resource interface (e.g., the interfaces illustrated in FIG. 4-6) on a display device/component of the user device 104.

FIG. 2 illustrates a schematic representation 200 of system components for managing source entities for recipient entities, and evaluating data associated with source entities to determine resource providers for recipient entities (e.g., managing suppliers for healthcare organization, in accordance with one embodiment of the invention). Specifically, FIG. 2 generally depicts a computer system and its components associated with the processing application module 144 of the processing system 106, which is structured for managing source entities for recipient entities, and evaluating data associated with source entities to determine resource providers for recipient entities in accordance with one of the disclosed embodiments. The processing application module 144 may also be referred to as a computer-executable platform.

As illustrated by FIG. 2, the processing application module 144 comprises a source entity evaluation system 210 (also referred to as a supplier evaluation system 210) communicating with the plurality of source entity systems 108 (also referred to as a supplier entity system(s) 108)and an entity terminal system 230 (also referred to as a business terminal system 230) via a communication network 240. In some embodiments, the communication network 240 is a part of the network 101 of FIG. 1.

Typically, the source entity evaluation system 210 (supplier evaluation system 210) comprises a category file database 211 storing a plurality of category files, and a source entity management database 212 (also referred to as a supplier management database 212) storing source entity data such as a plurality of complexities associated with the source entities (suppliers). The source entity evaluation system 210 (supplier evaluation system 210) further comprises a benchmarking module 213 structured for storing a plurality of source entity/supplier quality standards, and a resource value analysis module 214 (cost analysis module 214) configured to generate maturity amount resource values corresponding to the source entities (suppliers).

As shown further in FIG. 2, the source entity evaluation system 210 (supplier evaluation system 210) further comprises a predictive plan database 215 structured to store a plurality of customer's electronic records (e.g., contracts) provided by the entity terminal systems 230 (business terminal systems 230). Here, the entity terminal system 230 (business terminal system 230) is an organization system based upon the healthcare organization. For example, the healthcare organization may be selected from a group consisted of hospitals, emergency centers, private clinics, and rehabilitation facilities. In other words, the business's users of the entity terminal system 230 (business terminal systems 230) can set up an account and upload their business' electronic records (contracts) to the source entity evaluation system 210 (supplier evaluation system 210) via the predictive plan database 215. Alternatively, the users may log into their accounts and update the latest changes in their current selected electronic records (contracts).

The present invention also features a negotiate module 216 structured for communicating with the plurality of source entity systems 108 (supplier entity system(s) 108) and the entity/business terminal system 230 through the communication network 240. Here, the negotiate module 216 may receive source entity/supplier responses in response to category files provided by the plurality of source entity systems 108 (supplier entity system(s) 108). In addition, the users from the entity/business terminal system 230 can negotiate the resource values or costs of the resources/services in their electronic records/contracts via the negotiate module 16. Here, the negotiate module 216 may be designed as a real-time communication platform which allows the source entities/suppliers and the users to directly and operatively communicate thereon. In some embodiments, in response to the communication, the communications in the negotiate module 216 are written as an objective format. In this way, the source entity/supplier may directly update the quotations of their services, and the users from the business terminal system 230 may select a response “yes” or “no” for their source/supplier entities' quotations. Alternatively, the users from the entity/business terminal system 230 may provide a predetermined value for the source/supplier entity's services to the source entities/suppliers. If the source entity/supplier answers “yes” for that predetermined value, the electronic record/contract management module 217 may automatically update the electronic records/contracts from the users in accordance with the predetermined value. However, if the source entity (supplier) answers “no” for that predetermined value, the communication may restart. Therefore, the communication in the negotiate module 216 may repeat again and again until the source entities (suppliers) and the users have the agreement about the services' contracts.

In another embodiment, the source entity evaluation system 210 (supplier evaluation system 210) further comprises an interface 218 enabling remote access through the communication network 240. The interface 218 enables the source entities (suppliers) to electronically download the electronic records/contracts, which may be in the form of executable code or a protected data file. The interface 218 may allow the source entities (suppliers) to electronically upload the supplier's responses to the source entity evaluation system 210 (supplier evaluation system 210). In addition, the interface 218 may allow the source entities (suppliers) to selectively access the source entity management database 212 (supplier management database 212) in order to receive the information of existing vendor performance reports and product updates, as well as download the specifications relevant to the particular contracts.

The present system also features a benchmarking module 213 having a plurality of source entity/supplier quality standards provided by the source entity/supplier system(s) 108 and the entity/business terminal system 230. Here, not only the source entities (suppliers), but also the users of the recipient entity systems 110 (e.g., healthcare organization) may update the source entity/supplier quality standards. The source entity (supplier) quality standards may comprise estimated costs, service level, and degrees of satisfaction of the service. In one embodiment, the amount values are calculated by the amount of expenses from each of the source entities (suppliers). The resource value/cost analysis module 214 may calculate the amount values and evaluate them in accordance with the source entity/supplier quality standards to generate the maturity amount values.

In yet another embodiment, as shown further in FIG. 2, the source entity evaluation system 210 (supplier evaluation system 210) further comprises an interactive evaluation module 219 structured to determine a compliance supplier associated with the maturity customer's contracts in accordance with the maturity amount values.

FIG. 3 illustrates a high level process flow 300 for managing source entities for recipient entities, and evaluating data associated with source entities to determine resource providers for recipient entities (e.g., managing suppliers for healthcare organization, in accordance with one embodiment of the invention). As indicated previously, some or all of the steps of the process flow 300 may be performed by the system 106 (“the system”) in conjunction with the processing application module 144, via the network 140, as described with respect to FIG. 2.

As illustrated by block 310 of FIG. 3, the system may input user information to a source entity evaluation system via an interface.

Next, the system may load a customer's electronic record having a plurality of category files from a predictive plan database, as indicated by block 320.

Next, at block 330, the system may calculate amount values from the customer's electronic record in accordance with source entity quality standards provided by a benchmarking module to generate maturity amount values.

In some embodiments, as illustrated by block 340, the system is configured to present a maturity electronic record via an electronic record management module.

Accordingly, the maturity contract is presented with a screen that provides the user for editing the maturity contract by a word-processing based system. In another embodiment, the method further comprises the steps of: updating customer's contracts by a supplier system via a communication network; updating the category files in accordance with a supplier management database, and negotiating the amount of values with the source entities (suppliers) via a negotiate module.

In yet another embodiment, the method further comprises the steps of: computing a percentage of the amount values in the category file and identify a critical category file with a larger amount of percentage; updating customer's contracts provided by a business terminal system to the predictive plan; and initiating an interactive evaluation section to select a compliance source entity (supplier) for the category file.

FIG. 4 illustrates a graphical interface 400 of a category file 410 of the system of FIG. 2. As illustrated by FIG. 4, each of the category files 410 can comprise a plurality of specification items 412. Each of the specification items 412 can determine an amount value which can be determined as a number or words. In this ways, the specification items 412 can be selected from a group comprising a category name 412a, a spend with a specific term 412b, a projected saving 412c, a projected dollar saving 412d, a largest current source entity (supplier) 412e, a contract expiration date 412f, an existing source entity (supplier) 412g, and a recommendation source entity (supplier) 412g. It should be understood that the above-described specification items are exemplary and any other specification items can be adopted in various embodiments of this disclosure.

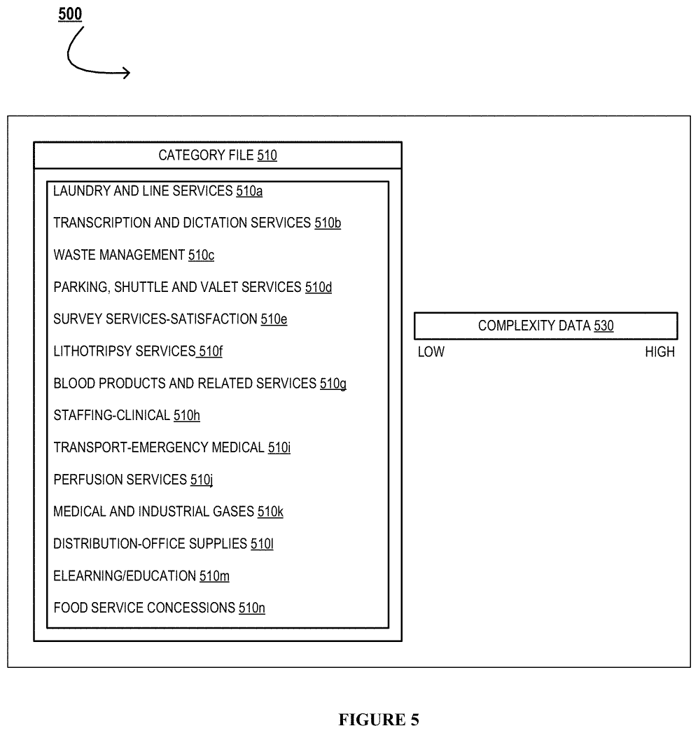

FIG. 5 illustrates a graphical interface 500 of the complexities for the category file of the system of FIG. 2. As illustrated by FIG. 5, the category files 510 may comprise laundry and line services 510a, transcription and dictation services 510b, waste management 510c, parking, shuttle and valet services 510d, survey services-satisfaction 510e, lithotripsy services 510f, blood products and related services 510g, staffing-clinical 510h, transport-emergency medical 510i, perfusion services 510j, medical and industrial gases 510k, distribution-office supplies 510l, eLearning/Education 510m, and/or food service concessions 510n. FIG. 5 also illustrates complexity data 530 on a scale from low to high. It should be understood that the above-described category files are exemplary and any other category files can be adopted in various embodiments of this disclosure.

The specification items are based upon the source entities (suppliers) provided by the source entity/supplier system(s) 108. Here, the source entity/supplier system(s) 108 may comprise a plurality of complexities (complexity data 530) corresponding to the source entities/suppliers. The complexities/complexity data 530 may comprise locations, services levels, service types, transaction costs, service terms, special discounts, and potential risks. In other words, the source entity/supplier system(s) 108 may update the complexities/complexity data 530 corresponding to each of the source entities/suppliers.

As described previously, the benchmarking module 213 comprises a plurality of source entity/supplier quality standards provided by the source entity/supplier system(s) 108 and the entity/business terminal system 230. Here, not only the source entities (suppliers), but also the users of the recipient entity systems 110 (e.g., healthcare organization) may update the source entity/supplier quality standards. The source entity (supplier) quality standards may comprise estimated costs, service level, and degrees of satisfaction of the service.

In one embodiment, the amount values are calculated by the amount of expenses from each of the source entities (suppliers). The resource value/cost analysis module 214 may calculate the amount values and evaluate them in accordance with the source entity/supplier quality standards to generate the maturity amount values.

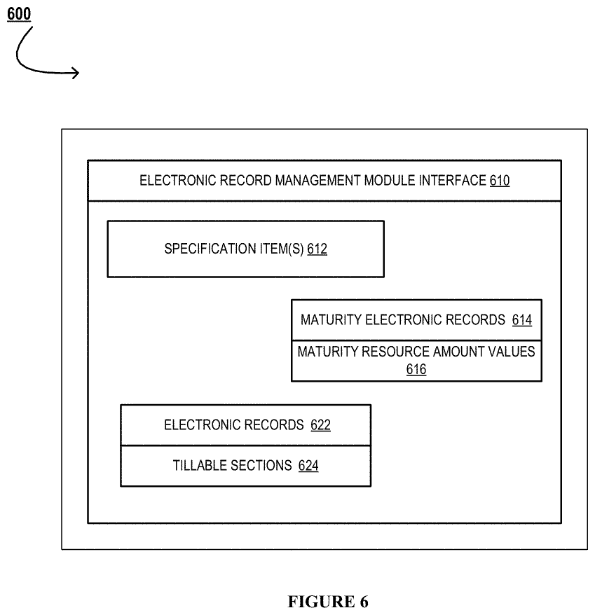

FIG. 6 illustrates a graphical interface 600 of electronic record/contract management module of the system of FIG. 2, showing the maturity amount values and the tillable sections. FIG. 6 illustrates an interface 610 of the electronic record/contract management module 217 of FIG. 2 showing the details of the specification items 612 in the electronic records/contracts. The electronic record/contract management module 217 stores a plurality of maturity electronic records/ contracts 614 having the plurality of category files having the maturity resource amount values 616. In this way, the users may select the best contracts for their requested services and record their selected services via the contract management 17 of the source entity evaluation system 210 (supplier evaluation system 210).

As discussed previously, the interactive evaluation module 219 is structured to determine a compliance supplier associated with the maturity customer's contracts in accordance with the maturity amount values. For example, in the embodiment of FIG. 6, the electronic records/contracts 622 shown in the electronic record/contract management module interface 610 may comprise a plurality of tillable sections 624, which allow the user to fill out customized amounts or terms. Indeed, the customized amount and terms may be analyzed by the interactive evaluation module 219 to generate corresponding specification terms, such as a saving percentage, a category spend, a projected saving, or the compliance source entities (suppliers). In other words, the interactive evaluation module 219 may be configured to recommend the best source entities (suppliers) and estimate the costs of the services provided by that source entity (supplier) based upon the customized inputted amounts and terms.

In addition, the interactive evaluation module 219 also can feature to compute a percentage of the amount values in the category file, wherein a category has a large amount of percentage may be identified as a critical category file, and the interactive evaluation module 219 may recommend that critical category file to the customer in order to consolidate the source entities (suppliers).

In some embodiments, the interface 610 may also comprise an interface associated with the predictive plan database 215. In other words, this interface may comprise a graphical representation of a predictive plan of the system.

The user also may fill out a request for proposal (RFP) or a request for information (RFI) forms for customers in electronic format to enable the user to respond to the questions, wherein the questions may be general or specific questions about the source entity's (supplier's) capabilities. In this way, the source entity (supplier) may also provide REP/RFI forms on their sites, so the supplier management database may update the REP/RFI on the source entities/suppliers' sites in a synchronized manner. Therefore, the cost analysis module may analyze the REP/RFI forms from the customers in accordance with the REP/RFI forms form the source entities (suppliers), during which point the source entities/suppliers' responses are appropriately compiled into the appropriate portion of the REP/RFI forms from the customers.

Similarly, while operations and/or methods may be depicted in the drawings in a particular order, this should not be understood as requiring that such operations be performed in the particular order shown or in sequential order, or that all illustrated operations and/or method steps be performed, to achieve desirable results. In certain circumstances, multitasking and parallel processing may be advantageous.

As will be appreciated by one of ordinary skill in the art, the present invention may be embodied as an apparatus (including, for example, a system, a machine, a device, a computer program product, and/or the like), as a method (including, for example, a business process, a computer-implemented process, and/or the like), or as any combination of the foregoing. Accordingly, embodiments of the present invention may take the form of an entirely software embodiment (including firmware, resident software, micro-code, and the like), an entirely hardware embodiment, or an embodiment combining software and hardware aspects that may generally be referred to herein as a “system.” Furthermore, embodiments of the present invention may take the form of a computer program product that includes a computer-readable storage medium having computer-executable program code portions stored therein.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

It will be understood that any suitable computer-readable medium may be utilized. The computer-readable medium may include, but is not limited to, a non-transitory computer-readable medium, such as a tangible electronic, magnetic, optical, infrared, electromagnetic, and/or semiconductor system, apparatus, and/or device. For example, in some embodiments, the non-transitory computer-readable medium includes a tangible medium such as a portable computer diskette, a hard disk, a random access memory (RAM), a read-only memory (ROM), an erasable programmable read-only memory (EPROM or Flash memory), a compact disc read-only memory (CD-ROM), and/or some other tangible optical and/or magnetic storage device. In other embodiments of the present invention, however, the computer-readable medium may be transitory, such as a propagation signal including computer-executable program code portions embodied therein.

It will also be understood that one or more computer-executable program code portions for carrying out the specialized operations of the present invention may be required on the specialized computer include object-oriented, scripted, and/or unscripted programming languages, such as, for example, Java, Perl, Smalltalk, C++, SAS, SQL, Python, Objective C, and/or the like. In some embodiments, the one or more computer-executable program code portions for carrying out operations of embodiments of the present invention are written in conventional procedural programming languages, such as the “C” programming languages and/or similar programming languages. The computer program code may alternatively or additionally be written in one or more multi-paradigm programming languages, such as, for example, F#.

Embodiments of the present invention are described above with reference to flowcharts and/or block diagrams. It will be understood that steps of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

It will also be understood that the one or more computer-executable program code portions may be stored in a transitory or non-transitory computer-readable medium (e.g., a memory, and the like) that can direct a computer and/or other programmable data processing apparatus to function in a particular manner, such that the computer-executable program code portions stored in the computer-readable medium produce an article of manufacture, including instruction mechanisms which implement the steps and/or functions specified in the flowchart(s) and/or block diagram block(s). The computer program product comprises a non-transitory computer-readable storage medium having computer-executable instructions.

The one or more computer-executable program code portions may also be loaded onto a computer and/or other programmable data processing apparatus to cause a series of operational steps to be performed on the computer and/or other programmable apparatus. In some embodiments, this produces a computer-implemented process such that the one or more computer-executable program code portions which execute on the computer and/or other programmable apparatus provide operational steps to implement the steps specified in the flowchart(s) and/or the functions specified in the block diagram block(s). Alternatively, computer-implemented steps may be combined with operator and/or human-implemented steps in order to carry out an embodiment of the present invention.

Many alterations and modifications may be made by those having ordinary skill in the art without departing from the spirit and scope of the disclosed embodiments. Therefore, it must be understood that the illustrated embodiments have been set forth only for the purposes of example and that it should not be taken as limiting the embodiments as defined by the following claims. For example, notwithstanding the fact that the elements of a claim are set forth below in a certain combination, it must be expressly understood that the embodiment includes other combinations of fewer, more or different elements, which are disclosed herein even when not initially claimed in such combinations.

Thus, specific embodiments and applications of a method for managing source entities (suppliers) for healthcare organizations have been disclosed. It should be apparent, however, to those skilled in the art that many more modifications besides those already described are possible without departing from the disclosed concepts herein. The disclosed embodiments, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the embodiments. In addition, where the specification and claims refer to at least one of something selected from the group consisting of A, B, C . . . and N, the text should be interpreted as requiring at least one element from the group which includes N, not A plus N, or B plus N, etc.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims, therefore, include not only the combination of elements which are literally set forth but all equivalent structure, material or acts for performing substantially the same function in substantially the same way to obtain substantially the same result. In this sense, it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub combination or variation of a sub combination.

What is claimed is:

1. A computer system for managing source entities for recipient entities comprising healthcare organizations, the system comprising:
a source entity evaluation system, comprising;
a category file database storing a plurality of category files having a plurality of amount values;
a source entity management database storing a plurality of complexities data for the source entities and configured to received updated complexities data from a source entity system;
a benchmarking module storing a plurality of source entity quality standards to be bases of the amount values and configured to generate maturity amount values in accordance with the amount values and the source entity quality standards;
a cost analysis module configured to calculate amounts of expenses for each of the source entities to generate the amount values; and
an electronic record management module storing a plurality of maturity electronic records having the plurality of category files having the maturity amount values.

2. The computer system, as recited in claim 1, further comprising the source entity system providing category files to the source entity evaluation system through a communication network.

3. The computer system, as recited in claim 2, further comprising an entity terminal system providing user's information and user's electronic records to the source entity evaluation system through the communication network, wherein the electronic records comprise contracts.

4. The computer system, as recited in claim 3, wherein the source entity evaluation system further comprises a negotiate module communicating with the source entity system and the entity terminal system through the communication network, wherein the negotiate module can receive source entity responses in response to category files provided by the source entity system.

5. The computer system, as recited in claim 4, wherein the source entity evaluation system further comprises a predictive plan database to store a plurality of customer's electronic records provided by the entity terminal systems.

6. The computer system, as recited in claim 5, wherein the complexities data is selected from the group consisting of locations, services levels, service types, transaction costs, service terms, special discounts, and potential risks.

7. The computer system, as recited in claim 5, wherein the predictive plan supplies the customer's electronic records to the cost analysis module, the amount of values being calculated in accordance with the source entity quality standards in the customer's electronic records.

8. The computer system, as recited in claim 7, wherein the source entity evaluation system further comprises an interface enabling remote access through the communication network.

9. The computer system, as recited in claim 8, wherein the source entity evaluation system further comprises an interactive evaluation module to identify a compliance source entity associated with the maturity electronic records in accordance with the maturity amount values.

10. A computerized method for managing source entities for recipient entities comprising healthcare organizations, the method comprising:
inputting user information to a source entity evaluation system via an interface;
loading a customer's electronic record having a plurality of category files from a predictive plan database;
calculating amount values from the customer's electronic record in accordance with source entity quality standards provided by a benchmarking module to generate maturity amount values; and
presenting a maturity electronic record by an electronic record management module.

11. The method of claim 10, further comprising the step of updating customer's electronic records by a source entity system via a communication network, wherein the electronic records comprise contracts.

12. The method of claim 11, further comprising the step of updating the category files in accordance with a source entity management database.

13. The method of claim 12, further comprising the step of negotiating the amount values with the source entities via a negotiate module.

14. The method of claim 13, further comprising the step of computing a percentage of the amount values in the category file and identify a critical category file with a larger amount of percentage.

15. The method of claim 14, further comprising the step of uploading customer's electronic records provided by an entity terminal system to the predictive plan.

16. The method of claim 15, wherein the computing step comprises the step of initiating an interactive evaluation section to select a compliance source entity for the category file.
